(19) United States
(12) Patent Application Publication
(10) Pub. No.: US 2025/0269151 A1
(43) Pub. Date: Aug. 28, 2025

(54) BALLOON FOR BALLOON CATHETER AND BALLOON CATHETER INCLUDING SAME

(71) Applicant: KANEKA CORPORATION, Osaka (JP)

(72) Inventors: Yoshinori Nakano, Osaka (JP); Kazuki Matsufuji, Osaka (JP); Masahiro Kojima, Osaka (JP); Masato Tsueda, Osaka (JP)

(73) Assignee: KANEKA CORPORATION, Osaka (JP)

(21) Appl. No.: 19/207,761

(22) Filed: May 14, 2025

Related U.S. Application Data
(63) Continuation of application No. PCT/JP2023/040824, filed on Nov. 13, 2023.

(30) Foreign Application Priority Data
Nov. 17, 2022 (JP) 2022-184271

(51) Int. Cl. A61M 25/10

(52) U.S. Cl. CPC A61M 25/10; A61M 2025/1075

(57) ABSTRACT

A balloon for a balloon catheter that is more likely to follow the inner wall of the stenosis and readily come into contact with the inner wall, thereby improving the performance of dilation of the stenosis, is provided. The balloon includes a balloon film including a first layer and a second layer having a Shore D hardness higher than that of the first layer, and the second layer is located outside the first layer. A variation rate ((|Tx−Ta|/Ta)×100) of a film thickness Tx of the balloon film with respect to an average film thickness Ta is 15% or less, an average film thickness T1a of the first layer is 2 μm or more, and a variation rate ((|T1x−T1a|/T1a)×100) of a film thickness T1x of the first layer with respect to the average film thickness T1a is 20% or more.

### TECHNICAL FIELD

One or more embodiments of the present invention relate to a balloon for a balloon catheter and a balloon catheter including the same.

### BACKGROUND

Diseases such as angina pectoris and myocardial infarction are caused by the formation of stenosis in the inner wall of a blood vessel. One treatment method for these diseases is angioplasty, such as percutaneous transluminal coronary angioplasty (PTCA) or percutaneous transluminal angioplasty (PTA), in which a balloon catheter is used to dilate the stenosed site. Angioplasty is a minimally invasive therapy that does not require thoracotomy like bypass surgery, and it is widely performed.

In angioplasty, the distal end of a balloon provided at a distal portion of a balloon catheter is inserted from a puncture site such as the femoral artery or the brachial artery, and the balloon is delivered through a vascular lumen to a lesion site by manipulation from the proximal side of the balloon catheter. Accordingly, balloon catheters having a balloon with improved ease of insertion, or a balloon provided with protrusions capable of dilating the stenosed site, have been developed. For example, Patent document 1 discloses a balloon catheter in which a balloon formed of a single material over the entire balloon is reduced in diameter to improve ease of insertion, and Patent document 2 discloses a balloon catheter in which protrusions having greater rigidity than the balloon wall are provided with a dilating function.

### PATENT DOCUMENT

Patent document 1: JP 2014-155657 A
Patent document 2: US 2016/0128718 A1

However, in the above conventional balloon catheter, when the balloon is inflated at a site having a complex inner lumen shape such as a stenosis after being delivered to a lesion site, there has been a problem in that the balloon cannot sufficiently come into contact with the inner wall of the lesion site, resulting in inefficient dilation of the stenosis.

### SUMMARY

In view of the above circumstances, a balloon for a balloon catheter that, when the balloon is inflated at a stenosis, is more likely to follow the inner wall of the stenosis and readily come into contact with the inner wall, thereby improving the performance of dilation of the stenosis, and a balloon catheter comprising the same, are provided.

A balloon for a balloon catheter according to one or more embodiments of the present invention, which has addressed the above, is as follows.
[1] A balloon for a balloon catheter having a longitudinal axis direction, a radial direction, and a circumferential direction, comprising: a balloon film including a first layer and a second layer composed of a material having a Shore D hardness higher than that of the first layer, the first layer and the second layer being disposed over the entire 360° in the circumferential direction; and the second layer being located on an outer side of the first layer in the radial direction, wherein a variation rate ((|Tx−Ta|/Ta)×100) of a film thickness Tx of the balloon film at any position x in the circumferential direction with respect to an average film thickness Ta of the balloon film in a cross-section perpendicular to the longitudinal axis direction is 15% or less; an average film thickness T1a of the first layer is 2 μm or more; and the balloon has, in a cross-section perpendicular to the longitudinal axis direction, a position X1 at which a variation rate ((|T1x−T1a|/T1a)×100) of a film thickness T1x of the first layer at the position X1 in the circumferential direction with respect to the average film thickness T1a of the first layer is 20% or more.
[2] The balloon for a balloon catheter according to [1], wherein a cross-section perpendicular to the longitudinal axis direction has a first sector, a second sector, and a third sector obtained by dividing the 360° in the circumferential direction into three sectors of 120° each, and the balloon has, in each sector, a position X1 at which a variation rate ((|T1x−T1a|/T1a)×100) of the film thickness T1x of the first layer at the position X1 in the circumferential direction with respect to the average film thickness T1a of the first layer is 20% or more.
[3] The balloon for a balloon catheter according to [2], wherein, in each of the first sector, the second sector, and the third sector, the balloon has: at least one first-layer-rich portion in which the film thickness T1x of the first layer is more than 50% of a film thickness Tx1 of the balloon film at the position X1 in the circumferential direction; and at least one second-layer-rich portion in which a film thickness T2x of the second layer at a position X2 in the circumferential direction is more than 50% of a film thickness Tx2 of the balloon film at the position X2.
[4] The balloon for a balloon catheter according to [3], wherein, in the circumferential direction of each of the sectors, the at least one first-layer-rich portion comprises a plurality of first-layer-rich portions, and the at least one second-layer-rich portion comprises a plurality of second-layer-rich portions; a total range in which the first-layer-rich portions are arranged is 30° or more out of the 120° in the circumferential direction of each sector; a total range in which the second-layer-rich portions are arranged is 30° or more out of the 120° in the circumferential direction of each sector; and each of the first-layer-rich portions and each of the second-layer-rich portions are alternately arranged in the circumferential direction.
[5] The balloon for a balloon catheter according to any one of [1] to [4], wherein the balloon includes: at least one first-layer-rich portion in which the film thickness T1x of the first layer is more than 50% of a film thickness Tx1 of the balloon film at the position X1 in the circumferential direction; and at least one second-layer-rich portion in which a film thickness T2x of the second layer at a position X2 in the circumferential direction is more than 50% of a film thickness Tx2 of the balloon film at the position X2; in the circumferential direction, the at least one first-layer-rich portion comprises a plurality of first-layer-rich portions, and the at least one second-layer-rich portion comprises a plurality of second-layer-rich portions; a range in which each of the first-layer-rich portions is arranged and a range in which each of the second-layer-rich portions is arranged is 15° or more, respectively, out of 360° in the circumferential direction of the balloon film; and each of the first-layer-rich portions and each of the second-layer-rich portions are alternately arranged in the circumferential direction.

One or more embodiments of the present invention also provide the following. [6] A balloon catheter comprising the balloon for a balloon catheter according to any one of [1] to [5].

According to the above balloon for a balloon catheter and the balloon catheter comprising the same, it is possible to provide a balloon for a balloon catheter and a balloon catheter comprising the same that, when the balloon is inflated at a stenosis, is more likely to follow the inner wall of the stenosis and readily come into contact with the inner wall, thereby improving the performance of dilation of the stenosis.

### BRIEF DESCRIPTION OF THE DRAWINGS

FIG. 1 is a side view of a balloon catheter according to one or more embodiments of the present invention.

FIG. 2 is a cross-sectional view taken along line II-II of the balloon catheter shown in FIG. 1.

FIG. 3 is an enlarged view of the first sector of the cross-sectional view shown in FIG. 2.

FIG. 4 is a variation of the cross-sectional view taken along line II-II.

FIG. 5 is a perspective view of a parison before biaxial stretching, according to one or more embodiments of the present invention.

FIG. 6 is a cross-sectional view taken along line VI-VI of the parison shown in FIG. 5.

FIG. 7 is a cross-sectional view perpendicular to the longitudinal axis direction of a parison mold used for manufacturing the parison shown in FIG. 6.

FIG. 8 is a longitudinal cross-sectional view of a mold according to one or more embodiments of the present invention.

FIG. 9 is a cross-sectional view taken along line IX-IX in FIG. 8.

### DETAILED DESCRIPTION

Hereinafter, one or more embodiments of the present invention will be described based on the following embodiments, however, the present invention is not limited by the following embodiments and can be altered in design within a scope in compliance with the intent described above and below, and all the changes are to be encompassed within a technical scope of the present invention. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of one or more embodiments of the present invention, the dimensions may differ from the actual dimensions in some cases.

### 1. Balloon for Balloon Catheter

A balloon for a balloon catheter according to one or more embodiments of the present invention has a longitudinal axis direction, a radial direction, and a circumferential direction; and has a balloon film including a first layer and a second layer composed of a material having a Shore D hardness higher than that of the first layer. The first layer and the second layer are disposed over the entire 360° in the circumferential direction; the second layer is located on an outer side of the first layer in the radial direction. A variation rate ((|Tx−Ta|/Ta)×100) of a film thickness Tx of the balloon film at any position x in the circumferential direction with respect to an average film thickness Ta of the balloon film in a cross-section perpendicular to the longitudinal axis direction is 15% or less; an average film thickness T1a of the first layer is 2 μm or more; and the balloon has, in a cross-section perpendicular to the longitudinal axis direction, a position x at which a variation rate ((|T1x−T1a|/T1a)×100) of a film thickness T1x of the first layer at the position x in the circumferential direction with respect to the average film thickness T1a of the first layer is 20% or more.

To dilate a stenosis using a balloon catheter, the balloon provided at the distal end of the balloon catheter is inserted into a vascular lumen and delivered to the stenosis, after which the balloon is inflated so that its outer wall comes into contact with the inner wall of the stenosis along the contour thereof, and the outer wall of the balloon is pressed against the inner wall of the stenosis. If there is a portion where the outer wall of the balloon does not follow the inner wall of the stenosis, the efficiency of dilation at that portion may be reduced even when the balloon is inflated. However, in the above balloon for a balloon catheter, the variation rate ((|Tx−Ta|/Ta)×100) of a film thickness Tx of the balloon film at any position x in the circumferential direction with respect to an average film thickness Ta of the balloon film is 15% or less, the balloon film includes a first layer and a second layer that has a higher Shore D hardness than the first layer, both arranged over the entire 360° in the circumferential direction, and the variation rate ((|T1x−T1a|/T1a)×100) of a film thickness T1x of the first layer with respect to the average film thickness T1a of the first layer is 20% or more, allowing the balloon film to have different stiffnesses in the circumferential direction. As a result, a portion of the balloon film having higher flexibility in the circumferential direction—specifically, a portion where the first layer, which has a lower Shore D hardness, is thicker-more easily follows the irregular part of the stenosis, so that the outer wall of the balloon readily comes into contact with the inner wall of the stenosis, thereby improving the performance of dilation of the stenosis. In addition, since the balloon film also has portions where the second layer with higher Shore D hardness is thick, the rigidity of the balloon can be maintained and its pressure resistance can be improved, and because the second layer is located radially outward of the first layer, it can also contribute to the improvement of the dilation performance at the stenosis.

In the present specification, the balloon for a balloon catheter may sometimes be simply referred to as “balloon.”

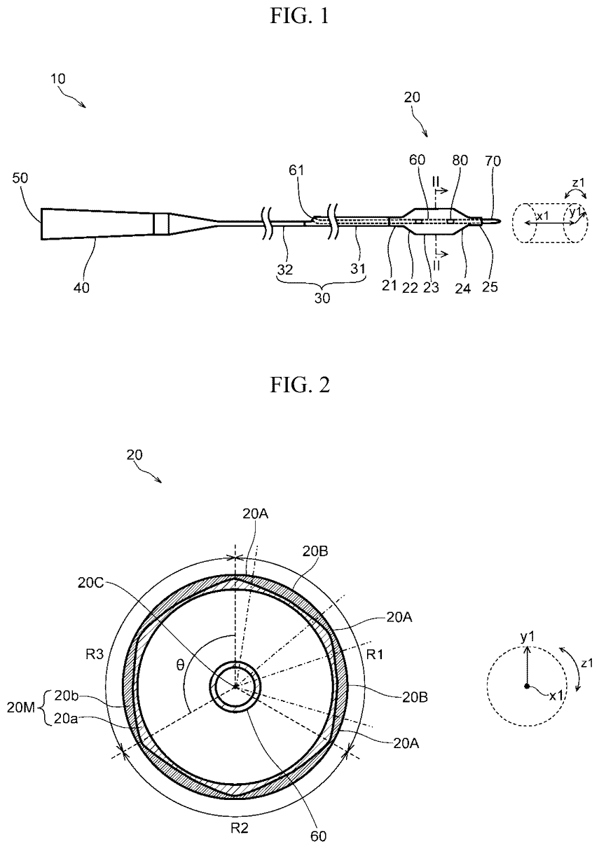

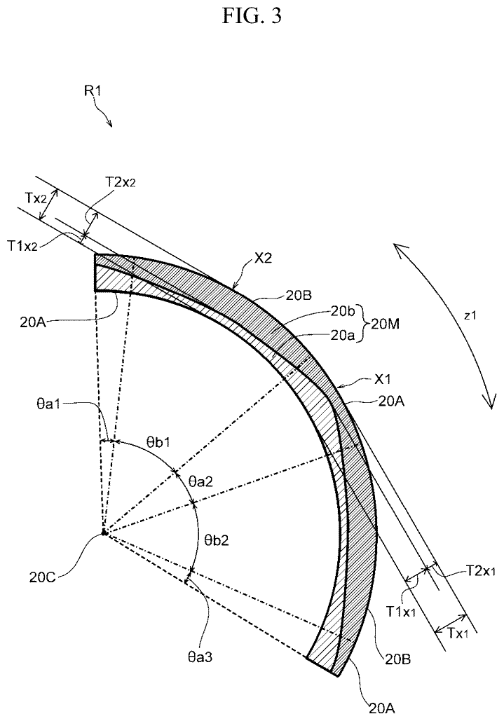

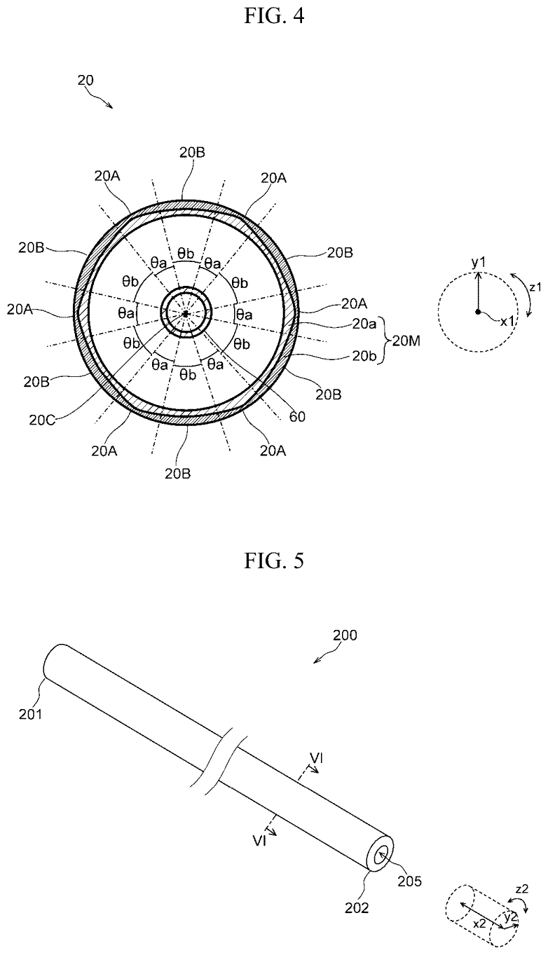

Hereinafter, a balloon for a balloon catheter according to one or more embodiments of the present invention will be described with reference to FIGS. 1 to 4. FIG. 1 is a side view of a balloon catheter according to one or more embodiments of the present invention. FIG. 2 is a cross-sectional view taken along line II-II of the balloon catheter shown in FIG. 1. In FIG. 2, the boundaries between the sectors are indicated by dotted lines, and the boundaries of the ranges in which the first-layer-rich portions and the second-layer-rich portions are arranged are indicated by alternate long and short dashed lines. FIG. 3 is an enlarged view of the first sector of the cross-sectional view shown in FIG. 2. In FIG. 3, the inner shaft is omitted, the boundaries between the sectors are indicated by dotted lines, and the boundaries of the ranges in which the first-layer-rich portions and the second-layer-rich portions are arranged are indicated by alternate long and short dashed lines. FIG. 4 is a cross-sectional view showing a variation of the cross-sectional view taken along line II-II of FIG. 2, in which the boundaries of the ranges in which the first-layer-rich portions and the second-layer-rich portions are arranged are indicated by alternate long and short dashed lines.

As shown in FIG. 1, a balloon 20 is provided at a distal portion of a balloon catheter 10. The balloon 20 is connected to a distal end portion of a shaft 30, and the balloon 20 can be inflated by introducing a fluid through a lumen of the shaft 30 and can be deflated by discharging the fluid. In order to control inflation and deflation of the balloon 20, a fluid may be introduced or discharged using an indeflator (balloon pressurizer). The fluid may be a pressurized fluid that is pressurized by a pump or the like. Details of the balloon catheter 10 will be described in the section “2. Balloon catheter.”

As shown in FIG. 2, the balloon 20 has a longitudinal axis direction x1, a radial direction y1 that connects the centroid 20C of the outer edge of the balloon 20 and a point on the outer edge in a cross-section perpendicular to the longitudinal axis direction x1, and a circumferential direction z1 that extends along the outer edge of the balloon 20 in the same cross-section. In the present specification, the direction toward the operator's side in the longitudinal axis direction x1 is referred to as the proximal side, and the opposite side, i.e., the direction toward the patient, is referred to as the distal side.

Members or portions other than the balloon 20 each have a longitudinal axis direction, a radial direction, and a circumferential direction. These directions may be the same as, or different from, the longitudinal axis direction x1, the radial direction y1, and the circumferential direction z1 of the balloon 20. However, for ease of understanding, in the present specification, all members and portions are described as having the same longitudinal axis direction, radial direction, and circumferential direction as the longitudinal axis direction x1, radial direction y1, and circumferential direction z1 of the balloon 20.

As shown in FIG. 2, the balloon 20 has a balloon film 20M including a first layer 20a and a second layer 20b made of a material having a higher Shore D hardness than the first layer 20a. The first layer 20a and the second layer 20b are arranged over the entire 360° in the circumferential direction z1, and the second layer 20b is located radially outward (in the radial direction y1) of the first layer 20a. The first layer 20a, which has a lower Shore D hardness and is continuously arranged over the entire 360° in the circumferential direction z1, contributes to improving the flexibility of the balloon 20. The second layer 20b, which has a higher Shore D hardness and is also continuously arranged over the entire 360° in the circumferential direction z1, ensures the rigidity of the balloon 20. Furthermore, the second layer 20b, which is located radially outside the first layer 20a and has a higher Shore D hardness, can contribute to improving the efficiency of dilation when the outer wall of the balloon 20 comes into contact with a stenosis.

In a cross-section perpendicular to the longitudinal axis direction x1, the variation rate of a film thickness Tx of the balloon film 20M at any position x in the circumferential direction z1 with respect to an average film thickness Ta of the balloon film 20M, that is, (|Tx−Ta|/Ta)×100 (%), where the absolute value of the difference between Tx and Ta is divided by Ta and multiplied by 100, is 15% or less. The average film thickness T1a of the first layer 20a is 2 μm or more. In a cross-section perpendicular to the longitudinal axis direction x1, the variation rate of a film thickness T1x of the first layer 20a at a position x in the circumferential direction z1 with respect to the average film thickness T1a of the first layer 20a, that is, (|T1x−T1a|/T1a)×100 (%), where the absolute value of the difference between T1x and T1a is divided by T1a and multiplied by 100, is 20% or more. While the variation rate of the film thickness of the balloon film 20M is 15% or less, the fact that the variation rate of the film thickness T1x of the first layer 20a, whose average thickness T1a is 2 μm or more, is 20% or more allows the balloon film 20M to have different stiffnesses depending on the position in the circumferential direction z1. As a result, a portion of the balloon film 20M having higher flexibility in the circumferential direction z1—that is, a portion where the thickness of the first layer 20a having a lower Shore D hardness is greater—can more easily follow a more irregular part of the stenosis, so that the outer wall of the balloon 20 readily comes into contact with the inner wall of the stenosis, thereby improving the performance of dilation of the stenosis. As a method for more efficiently exhibiting this effect, for example, the balloon 20 may be axially rotated after reaching the stenosis in a depressurized state lower than the balloon pressure used for treatment. In addition, since the balloon film 20M also includes portions in which the second layer 20b having a higher Shore D hardness is thick, the rigidity of the balloon 20 can be ensured and its pressure resistance can be improved.

The variation rate of a film thickness Tx of the balloon film 20M at any position x in the circumferential direction z1 with respect to the average film thickness Ta of the balloon film 20M may be 15% or less, 10% or less, 8% or less, or 5% or less. When the upper limit of the variation rate of the film thickness Tx in the circumferential direction z1 of the balloon film 20M falls within the above range, the outer surface of the balloon 20 can be configured so as not to protrude outward in the radial direction y1, thereby facilitating conformity of the outer wall of the balloon 20 to the inner lumen shape of a stenosis. In addition, since the balloon film 20M can be formed with a uniform thickness, this configuration may also be preferable in terms of the flexibility and durability of the balloon 20. Although the ideal lower limit of the variation rate of the film thickness Tx in the circumferential direction z1 of the balloon film 20M is 0%, it may practically be 1% or more, 2% or more, or 3% or more.

The Shore D hardness of the first layer 20a may be 20 or more, 25 or more, 30 or more, 35 or more, or 40 or more. The Shore D hardness of the first layer 20a may also be 70 or less, 65 or less, 60 or less, or 55 or less. The Shore D hardness of the second layer 20b may be more than 70, 72 or more, 74 or more, or 75 or more. The Shore D hardness of the second layer 20b may also be 90 or less, 85 or less, or 80 or less. When the Shore D hardnesses of the first layer 20a and the second layer 20b fall within the above ranges, the above-described effects can be achieved.

The Shore D hardness can be measured, for example, using a Type D durometer in accordance with JIS K6253-2:2012. The respective Shore D hardness values of the first layer 20a and the second layer 20b may be values of the materials before being formed into the balloon 20.

As the material of the second layer 20b, polyamide resins such as nylon 11 and nylon 12; polyester resins such as polyethylene terephthalate and polybutylene terephthalate; and polyurethane resins may be used. As the material of the first layer 20a, a thermoplastic elastomer may be used from the viewpoint of having a lower Shore D hardness, and for example, a polyamide elastomer such as a polyether block amide copolymer may be used.

As shown in FIGS. 2 to 4, the balloon 20 may be composed solely of the balloon film 20M. Alternatively, although not shown in the figures, the balloon 20 may include layers other than the balloon film 20M. When the balloon 20 includes layers other than the balloon film 20M, the balloon 20 may have a configuration in which the balloon film 20M has the above-described structure, and a second balloon film or a third balloon film is disposed on the inside or outside in the radial direction y1 of the balloon film 20M. Even in such a configuration, a balloon having multiple balloon films is included as the balloon 20 according to one or more embodiments of the present invention, provided that the balloon film 20M has the above-described structure.

The average film thickness Ta of the balloon film 20M may be 12 μm or more, 15 μm or more, or 20 μm or more. The average film thickness Ta may also be 60 μm or less, 50 μm or less, or 40 μm or less.

The average film thickness T1a of the first layer 20a is 2 μm or more and, within a range less than the average film thickness Ta of the balloon film 20M, may be, for example, 3 μm or more, 4 μm or more, 5 μm or more, or 6 μm or more, and may also be 55 μm or less, 50 μm or less, 40 μm or less, 30 μm or less, or 20 μm or less.

The balloon film 20M may be composed only of the first layer 20a and the second layer 20b, in which case the thickness of the second layer 20b is obtained by subtracting the thickness of the first layer 20a from the thickness of the balloon film 20M. The average film thickness of the second layer 20b may be 2 μm or more and, within a range less than the average film thickness Ta of the balloon film 20M, may be, for example, 3 μm or more, 4 μm or more, 5 μm or more, or 6 μm or more, and may also be 55 μm or less, 50 μm or less, 40 μm or less, 30 μm or less, or 20 μm or less.

In a cross-section perpendicular to the longitudinal axis direction x1, the variation rate of a film thickness T1x of the first layer 20a at a position x in the circumferential direction z1 with respect to the average film thickness T1a of the first layer 20a may be 20% or more, 30% or more, 40% or more, or 50% or more, and may also be 95% or less, 90% or less, or 80% or less. Since the balloon film 20M has a uniform film thickness below a predetermined level, the fact that the film thickness T1x of the first layer 20a has a variation rate within the above range causes the film thickness T2x of the second layer 20b to also have a similar variation rate, and, as shown in FIG. 2, the balloon film 20M can therefore have, depending on the position in the circumferential direction z1, a portion rich in the first layer 20a having a lower Shore D hardness and a portion rich in the second layer 20b having a higher Shore D hardness.

Each film thickness can be measured by observing a cross-section of the balloon 20 that is perpendicular to the longitudinal axis direction x1, and the observation can be performed using, for example, an optical microscope, from which the film thickness can be obtained based on the measured thickness in the observation image and the magnification. The average film thickness Ta of the balloon film 20M can be obtained by measuring the film thickness at 24 points spaced at 15° intervals over the full 360° in the circumferential direction z1 of the balloon film 20M and calculating the average of those 24 values. These 24 points can be defined as points on 24 radial lines drawn in the radial direction y1 from the centroid 20C of the outer edge of the balloon 20 to the outer edge such that the central angle θ (the smaller one) formed between adjacent lines is 15°. The number of measurement points for obtaining the average film thickness Ta is not limited to 24 and may be smaller or greater; however, it may be 8 or more.

The thickness may be measured by observing the balloon 20 in its inflated state. Although the method for maintaining the balloon 20 in the inflated state is not particularly limited, examples include a method in which the inflated balloon 20 is embedded in a curable resin for observation, a cross-section perpendicular to the longitudinal axis direction x1 is exposed, and the cross-section is observed. Alternatively, the balloon 20 may be directly observed without being embedded in the curable resin, or the balloon 20 in a deflated state may be observed as long as the central angle θ can be determined.

The film thickness Tx of the balloon film 20M at a position x in the circumferential direction z1 is not limited to the measurement points used to determine the average wall thickness Ta, and can be obtained by observing the film thickness at a position x in the circumferential direction z1 using the same method as described above.

The average film thickness T1a of the first layer 20a and the film thickness T1x of the first layer 20a at a position x in the circumferential direction z1 can also be measured in the same manner as in the case of the balloon film 20M. Since the first layer 20a and the second layer 20b are formed of different resins, the boundary between the layers can be observed under a microscope, allowing the thickness of each layer to be determined.

The variation rate of the film thickness T1x of the first layer 20a at a position x in the circumferential direction z1 with respect to the average film thickness T1a of the first layer 20a can be obtained by first determining the average film thickness T1a of the first layer 20a as described above and then, as shown in FIG. 3, measuring the film thickness T1x1 of the first layer 20a at a position X1 and substituting the measured value into the formula (|T1x1-T1a|/T1a)×100. As shown in FIG. 3, for example, the first layer 20a at the position X1 is thicker than at other positions, indicating that the film thickness of the first layer 20a at the position X1 varies toward the thicker side relative to the average film thickness T1a of the first layer 20a. Similarly, at a position X2, the film thickness T1x₂ of the first layer 20a can be measured, and the variation rate at the position X2 can be obtained using the formula (|T1x₂−T1a|/T1a)×100. At the position X2, the film thickness of the first layer 20a is thinner than at other positions, indicating that the film thickness at position X2 varies toward the thinner side relative to the average film thickness T1a. Thus, the film thickness of the first layer 20a may vary by 20% or more both toward the thicker side and the thinner side with respect to the average film thickness T1a, which makes it easier to vary the rigidity of the balloon film 20M depending on the position in the circumferential direction z1.

As shown in FIG. 2, in a cross-section perpendicular to the longitudinal axis direction x1, when the 360° in the circumferential direction z1 is divided into three sectors such that the central angle θ is 120°, and these sectors are defined as a first sector R1, a second sector R2, and a third sector R3, in each sector, the variation rate of the film thickness T1x of the first layer 20a at a position x in the circumferential direction z1 with respect to the average film thickness T1a of the first layer 20a, namely (|T1x−T1a|/T1a)×100, may be 20% or more. When the variation rate of the film thickness T1x of the first layer 20a is not less than a predetermined value in each sector, it becomes easier to vary the rigidity of the balloon film 20M across the entire circumferential direction z1, and the thick portions of the highly flexible first layer 20a can be distributed evenly in the circumferential direction z1 of the balloon film 20M. As a result, the outer wall of the balloon 20 can more easily conform to the inner wall of the stenosis, and it becomes easier for the outer wall of the balloon 20 to contact the inner wall of the stenosis and improve the dilation performance at the stenosis.

As shown in FIGS. 2 and 3, each of the first sector R1, the second sector R2, and the third sector R3 may include at least one first-layer-rich portion 20A, in which the film thickness T1x of the first layer 20a at a position x in the circumferential direction z1 is more than 50% of the film thickness Tx of the balloon film 20M at the same position x, and at least one second-layer-rich portion 20B, in which the film thickness T2x of the second layer 20b at a position x in the circumferential direction z1 is more than 50% of the film thickness Tx of the balloon film 20M at the same position x. The film thickness T1x of the first layer 20a and the film thickness Tx of the balloon film 20M may be compared at the same position x. Likewise, the film thickness T2x of the second layer 20b and the film thickness Tx of the balloon film 20M may be compared at the same position x. For example, as shown in FIG. 3, the film thickness T1x of the first layer 20a and the film thickness Tx of the balloon film 20M are compared at the position X1, and since the film thickness T1x₁ of the first layer 20a at the position X1 is more than 50% of the film thickness Tx1 of the balloon film 20M at the position X1, the position X1 can be determined to be the first-layer-rich portion 20A. Similarly, as shown in FIG. 3, the film thickness T2x of the second layer 20b and the film thickness Tx of the balloon film 20M are compared at the position X2, and since the film thickness T2x₂ of the second layer 20b at the position X2 is more than 50% of the film thickness Tx2 of the balloon film 20M at the position X2, the position X2 can be determined to be the second-layer-rich portion 20B.

Thanks to each sector including the first-layer-rich portion 20A and the second-layer-rich portion 20B, the rigidity of the balloon film 20M can be more significantly varied across the entire circumferential direction z1 of the balloon 20. Because the first-layer-rich portion 20A increases the flexibility of the balloon film 20M, the first-layer-rich portion 20A more easily deforms along the inner lumen shape of the stenosis, which makes contact between the outer wall of the balloon 20 and the inner wall of the stenosis easier. In addition, because each sector includes the first-layer-rich portion 20A, the flexible portions of the balloon film 20M are not biased toward any particular area in the circumferential direction z1, and therefore the need to axially rotate the balloon 20 in order to bring the outer wall of the balloon 20 into conformity with the inner wall of the stenosis can be reduced, enabling a safer procedure. The second-layer-rich portion 20B, having higher rigidity, contributes to an improvement in the strength and pressure resistance of the balloon 20.

As shown in FIG. 3, in the circumferential direction z1 of each of the sectors, the at least one first-layer-rich portion 20A may comprise a plurality of first-layer-rich portions 20A, and the at least one second-layer-rich portion 20B may comprise a plurality of second-layer-rich portions 20B; a total range in which the first-layer-rich portions 20A are arranged may be 30° or more out of the 120° in the circumferential direction z1 of each sector; a total range in which the second-layer-rich portions 20B are arranged may be 30° or more out of the 120° in the circumferential direction z1 of each sector; and each of the first-layer-rich portions 20A and each of the second-layer-rich portions 20B may be alternately arranged in the circumferential direction z1. In the circumferential direction z1 of each sector, the total range in which the second-layer-rich portions 20B are arranged may be greater than the total range in which the first-layer-rich portions 20A are arranged. With such a configuration, the second-layer-rich portions 20B can provide sufficient rigidity for the balloon 20, while the portions where the first-layer-rich portions 20A are arranged can function as flexible cushions, which facilitates conformity of the balloon 20 to the inner wall of the stenosis and makes it easier to improve the dilation performance. Alternatively, in the circumferential direction z1 of each sector, the total range in which the second-layer-rich portions 20B are arranged may be smaller than the total range in which the first-layer-rich portions 20A are arranged. This configuration allows the flexibility of the balloon 20 to be further enhanced, thereby making it easier for the balloon 20 to conform to the inner wall of the stenosis.

The range in which the first-layer-rich portions 20A and the second-layer-rich portions 20B are arranged can be defined, in a cross-section perpendicular to the longitudinal axis direction x₁, as the smaller angle formed by two line segments drawn from the centroid 20C of the outer edge of the balloon 20 to the outer edge at the respective ends of each portion. Since the plurality of first-layer-rich portions 20A and the plurality of second-layer-rich portions 20B are arranged in each sector, the total range in which the first-layer-rich portions 20A are arranged is the sum of the angles 0a1, 0a2, and 0a3, and the total range in which the second-layer-rich portions 20B are arranged is the sum of the angles 0b1 and 0b2. By ensuring that the total ranges in which the first-layer-rich portions 20A and the second-layer-rich portions 20B are arranged in each sector are 30° or more, respectively, the highly flexible first-layer-rich portions 20A and the highly rigid second-layer-rich portions 20B can be arranged over a sufficient angular range in each sector. As a result, varying the flexibility and rigidity of the balloon 20 across the entire circumferential direction z1 becomes easier.

As shown in FIG. 4, the balloon 20 may include at least one first-layer-rich portion 20A, in which the film thickness T1x of the first layer 20a at a position x in the circumferential direction z1 is more than 50% of the film thickness Tx of the balloon film 20M at the same position x, and at least one second-layer-rich portion 20B, in which the film thickness T2x of the second layer 20b at a position x in the circumferential direction z1 is more than 50% of the film thickness Tx of the balloon film 20M at the same position x. In the circumferential direction z1, the at least one first-layer-rich portion 20A may comprise a plurality of first-layer-rich portions 20A, and the at least one second-layer-rich portion 20B may comprise a plurality of second-layer-rich portions 20B; a range θa in which each of the first-layer-rich portions 20A is arranged and a range Ob in which each of the second-layer-rich portions 20B is arranged may be 15° or more, respectively, out of the 360° in the circumferential direction z1 of the balloon film 20M; and each of the first-layer-rich portions 20A and each of the second-layer-rich portions 20B may be arranged alternately in the circumferential direction z1.

In the circumferential direction z1 of the balloon 20, because each of the first-layer-rich portions 20A and each of the second-layer-rich portions 20B are arranged over a respective range that is equal to or greater than a predetermined angle, and because the first-layer-rich portions 20A and the second-layer-rich portions 20B are arranged alternately, sufficiently flexible portions and sufficiently rigid portions can exist alternately in the circumferential direction z1. As a result, making the outer wall of the balloon 20 conform to the inner wall of the stenosis becomes even easier.

The range θa over which each of the first-layer-rich portions 20A is arranged may be 20° or more, or 30° or more, and 60° or less, or 45° or less. The range Ob over which each of the second-layer-rich portions 20B is arranged may be 20° or more, or 30° or more, and 60° or less, or 45° or less. The number of the first-layer-rich portions 20A depends on the range θa in which each portion is arranged, and, for example, may be 12 or less, 9 or less, or 6 or less, and 3 or more, or 4 or more. The number of the second-layer-rich portions 20B depends on the range Ob in which each portion is arranged, and, for example, may be 12 or less, 9 or less, or 6 or less, and 3 or more, or 4 or more.

The range θa over which each of the first-layer-rich portions 20A is arranged may be greater than the range Ob over which each of the second-layer-rich portions 20B is arranged. By increasing the range θa, a larger portion of the balloon 20 having flexibility can be secured in the circumferential direction z1. Alternatively, the range θb over which each of the second-layer-rich portions 20B is arranged may be greater than the range θa over which each of the first-layer-rich portions 20A is arranged. By increasing the range θb, a larger portion of the balloon 20 having higher rigidity can be secured in the circumferential direction z1.

As shown in FIG. 1, the balloon 20 may have a proximal end and a distal end in the longitudinal axis direction x₁, and include a straight tubular part 23, a proximal tapered part 22 located proximally to the straight tubular part 23, a proximal sleeve part 21 located proximally to the proximal tapered part 22, a distal tapered part 24 located distally to the straight tubular part 23, and a distal sleeve part 25 located distally to the distal tapered part 24. The straight tubular part 23 may be substantially cylindrical, having an approximately constant diameter in the longitudinal axis direction x₁, although it may have varying diameters along the longitudinal axis direction x₁. The proximal tapered part 22 and the distal tapered part 24 may be formed in a substantially conical or truncated conical shape, tapering in diameter away from the straight tubular part 23. When the straight tubular part 23 has the largest diameter, the straight tubular part 23 can more readily contact the inner wall of a stenosis when the balloon 20 is inflated at the stenosis, thereby facilitating treatment such as dilation of the stenosis. In addition, since the proximal tapered part 22 and the distal tapered part 24 are reduced in diameter, the outer diameters of the proximal and distal ends of the balloon 20 can be reduced when the balloon 20 is deflated, which allows the step between the shaft 30 and the balloon 20 to be minimized, making the balloon 20 easier to insert into a body lumen.

The proximal tapered part 22, the straight tubular part 23, and the distal tapered part 24 may be portions that expand when a fluid is introduced into the balloon 20, whereas the proximal sleeve part 21 and the distal sleeve part 25 may not expand. This allows at least a part of the proximal sleeve part 21 to be fixed to the distal end of the shaft 30, and at least a part of the distal sleeve part 25 to be fixed to an inner shaft 60, which will be described later.

The first-layer-rich portions 20A and the second-layer-rich portions 20B may extend along the longitudinal axis direction x₁. The first-layer-rich portions 20A and the second-layer-rich portions 20B may extend in a straight line along the longitudinal axis direction x₁, or may extend helically.

The first-layer-rich portions 20A and the second-layer-rich portions 20B may extend throughout the entire length of the straight tubular part 23 along the longitudinal axis direction x₁. This facilitates conformity of the most expandable portion of the balloon 20 to the inner wall of a stenosis. In addition, the first-layer-rich portions 20A and the second-layer-rich portions 20B may also extend from the straight tubular part 23 toward the proximal tapered part 22 and/or the distal tapered part 24.

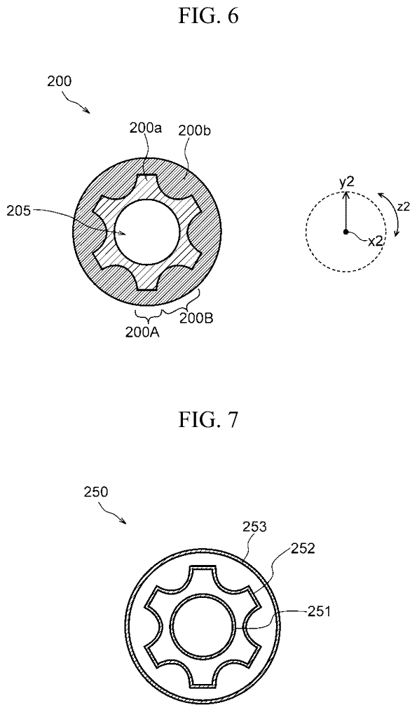

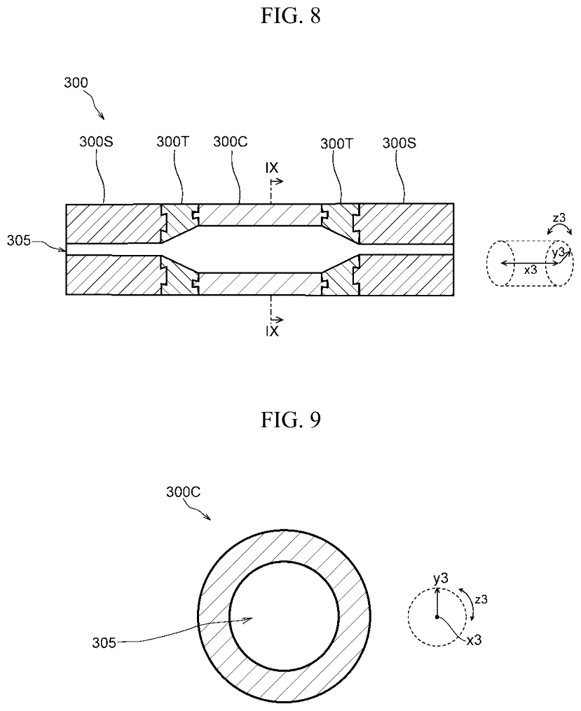

Next, a method for manufacturing the above-described balloon 20 according to one or more embodiments of the present invention will be described with reference to FIGS. 5 to 9. FIG. 5 is a perspective view of a parison before biaxial stretching, according to one or more embodiments of the present invention. FIG. 6 is a cross-sectional view taken along line VI-VI of the parison shown in FIG. 5 and represents a cross-sectional view of a parison used for manufacturing a balloon having the cross-section shown in FIG. 2. FIG. 7 is a cross-sectional view perpendicular to the longitudinal axis direction of a parison mold used for manufacturing the parison shown in FIG. 6. FIG. 8 is a longitudinal cross-sectional view of a mold used for stretching the parison, according to one or more embodiments of the present invention. FIG. 9 is a cross-sectional view taken along line IX-IX in FIG. 8.

First, a parison 200 is prepared. The parison 200 is made of a resin and is a cylindrical member having a lumen 205, as shown in FIG. 5. The parison 200 has a first end 201 and a second end 202, and extends in a longitudinal axis direction x₂ from the first end 201 to the second end 202. Similar to the balloon 20, the parison 200 has a radial direction y2 and a circumferential direction z2.

As shown in FIG. 6, the parison 200 includes a second layer 200b and a first layer 200a that is composed of a material having a lower Shore D hardness than that of the second layer 200b. The first layer 200a and the second layer 200b may be continuous over the entire circumferential direction z2. The materials forming the first layer 200a and the second layer 200b, as well as their Shore D hardness values, can be referred to in the descriptions of the resins forming the first layer 20a and the second layer 20b of the above-described balloon 20, and the descriptions of their Shore D hardness.

As shown in FIG. 6, in a cross-section perpendicular to the longitudinal axis direction x2, the first layer 200a may have a thick portion 200A and a thin portion 200B in the radial direction y2. From the viewpoint of the thickness of the second layer 200b, the second layer 200b may be thinner in the radial direction y2 at the thick portion 200A, and thicker at the thin portion 200B.

Such a parison 200 can be manufactured, for example, by extruding a resin using a parison mold 250 as shown in FIG. 7. As shown in FIG. 7, the parison mold 250 includes a first tubular member 251, a second tubular member 252, and a third tubular member 253. The first tubular member 251 may have a tubular shape so as to form the lumen 205 of the parison 200. The second tubular member 252 may have a gear-like shape so as to form the thick portion 200A and the thin portion 200B. The third tubular member 253 may have a tubular shape so as to form the parison 200 into a cylindrical configuration. By introducing a resin for forming the first layer 200a into the space between the outer surface of the first tubular member 251 and the inner surface of the second tubular member 252, and introducing a resin for forming the second layer 200b into the space between the outer surface of the second tubular member 252 and the inner surface of the third tubular member 253, the cylindrical parison 200 having the lumen 205, the thick portion 200A, and the thin portion 200B can be manufactured by extrusion.

The material constituting the parison mold 250 may be a metal, and the metal may be iron, copper, aluminum, or an alloy thereof. For example, stainless steel is a suitable alloy of iron, brass is a suitable alloy of copper, and duralumin is a suitable alloy of aluminum. From the standpoint of having sufficient strength and ease of processing, the parison mold 250 may be made of stainless steel.

By stretching the parison 200, the balloon 20 having the first layer 20a and the second layer 20b can be manufactured, in which the variation rate of the thickness of the first layer 20a in the circumferential direction z1 is equal to or greater than a predetermined value. At this time, a mold 300 as shown in FIG. 8 can be used. The mold 300 has a longitudinal axis direction x3, a radial direction y3, and a circumferential direction z3, and has a lumen 305 extending in the longitudinal axis direction x3 into which the parison 200 is inserted. A portion of the parison 200 in the longitudinal axis direction x2 may be positioned within the lumen 305 of the mold 300. The stretching of the parison 200 may be performed by blow molding the parison 200, or by biaxially stretching the parison 200.

The mold 300 may include, in the longitudinal axis direction x3: a mold straight tubular part 300C for forming the straight tubular part 23 of the balloon 20; two mold tapered parts 300T disposed on both sides of the mold straight tubular part 300C for forming the tapered parts of the balloon 20; and two mold sleeve parts 300S disposed on sides farther from the mold straight tubular part 300C than the mold tapered parts 300T for forming the sleeve parts of the balloon 20. Accordingly, the straight tubular part 23 of the balloon 20 can be formed by the mold straight tubular part 300C, the proximal tapered part 22 and the distal tapered part 24 can be formed by the mold tapered parts 300T, and the proximal sleeve part 21 and the distal sleeve part 25 can be formed by the mold sleeve parts 300S.

The mold 300 may be composed of a single member or of multiple members. As shown in FIG. 8, the mold 300 may be formed by connecting a plurality of mold components in the longitudinal axis direction x3. For example, the mold straight tubular part 300C, the mold tapered part 300T, and the mold sleeve part 300S may be separate mold components that are connected to one another in the longitudinal axis direction x3. The mold 300 may also be configured to be separable in the radial direction y, which facilitates the insertion of the parison 200 into the mold cavity 305. As shown in FIG. 8, each mold component may be joined by engaging with an adjacent mold component. Alternatively, although not shown in the figures, each adjacent mold component may be provided with a magnet and joined together by magnetic attraction.

As shown in FIG. 9, the mold cavity 305 of the mold 300 may be formed in a substantially circular shape. By placing the parison 200 in such a mold cavity 305 and introducing a fluid into the inner cavity 205 of the parison 200 to perform biaxial stretching, a balloon 20 having a thickness variation ratio within a predetermined range can be manufactured.

The mold 300 may be made of a metal, and the metal may be iron, copper, aluminum, or an alloy thereof. For example, stainless steel may be used as an iron alloy, brass may be used as a copper alloy, and duralumin may be used as an aluminum alloy. The mold 300 may be made of stainless steel from the standpoint of having sufficient strength and ease of processing.

### 2. Balloon Catheter

A balloon catheter 10 according to one or more embodiments of the present invention includes the above-described balloon 20 for a balloon catheter. As described in the section “1. Balloon for balloon catheter,” the balloon 20 is connected to a distal end portion of a shaft 30, as shown in FIG. 1.

FIG. 1 illustrates a so-called rapid-exchange type balloon catheter 10, which has a guidewire port 61 located midway between the distal and proximal sides of the shaft 30 and has an inner shaft 60 that functions as a guidewire lumen from the guidewire port 61 to the distal side of the shaft 30. The balloon catheter 10 may have a distal shaft 31 and a proximal shaft 32, with the distal shaft 31 and the proximal shaft 32 being separate members. The proximal end part of the distal shaft 31 may be connected to the distal end part of the proximal shaft 32, thereby forming the shaft 30 that extends from the balloon 20 to the proximal end part of the balloon catheter 10. Alternatively, a single shaft 30 may extend from the balloon 20 to the proximal end part of the balloon catheter 10, or the distal shaft 31 and the proximal shaft 32 may each consist of multiple tube members.

The shaft 30 may have a fluid flow path and a guidewire lumen inside. To configure the shaft 30 to have the internal fluid flow path and the guidewire lumen, for example, the inner shaft 60 located inside the shaft 30 may function as the guidewire lumen, and the space between the shaft 30 and the inner shaft 60 may function as the fluid flow path. In such a configuration, the inner shaft 60 may extend from the distal end of the shaft 30 and pass through the balloon 20, the distal side of the balloon 20 may be connected to the inner shaft 60, and the proximal side of the balloon 2 may be connected to the shaft 30.

The shaft 30 may be composed of resin, metal, or a combination of resin and metal. By using resin as the material for the shaft, flexibility and elasticity can be more easily imparted to the shaft 30. By using metal as the material for the shaft 30, the delivering performance of the balloon catheter 10 can be improved. Examples of resin used for the shaft 30 include polyamide-based resin, polyester-based resin, polyurethane-based resin, polyolefin-based resin, fluorine-based resin, polyvinyl chloride-based resin, silicone-based resin, natural rubber, and synthetic rubber. Any one of these may be used alone, or two or more may be used in combination. Examples of metal used for the shaft 30, include stainless steel such as SUS 304 and SUS 316, platinum, nickel, cobalt, chromium, titanium, tungsten, gold, Ni—Ti alloys, Co—Cr alloys, or combinations thereof. When the shaft 30 is composed of the distal shaft 31 and proximal shaft 32 as separate members, the distal shaft 31 may be, for example, made of resin, and the proximal shaft 32 may be made of metal. The shaft 30 may also have a layered structure using different materials or the same material.

The balloon 20 and the shaft 30 may be joined by adhesive bonding, welding, or by attaching a ring-shaped member at the point where the end of the balloon 20 and the shaft 30 overlap to swage them. Of these, the balloon 20 and the shaft 30 may be joined by welding. By welding the balloon 20 and the shaft 30, the bond between the balloon 20 and the shaft 30 is difficult to be released even when the balloon 20 is repeatedly inflated and deflated, easily increasing the strength of the bond between them.

The balloon catheter 10 may be provided with a tip member 70 at its distal end part. The tip member 70 may be provided at the distal end part of the balloon catheter 10 by being connected to the distal end part of the balloon 20 as a separate component from the inner shaft 60, or the inner shaft 60 extending distally beyond the distal end of the balloon 20 may function as the tip member 70.

A radiopaque marker 80 may be placed on the inner shaft 60 inside the balloon 20 at the location of the balloon 20 in the longitudinal axis direction x1, so that the position of the balloon 20 can be confirmed radiographically. The radiopaque marker 80 may be placed at a position corresponding to both ends of the straight tubular part 23 of the balloon 20, or may be placed at a position corresponding to the center of the straight tubular part 23 in the longitudinal axis direction x1.

A hub 40 may be provided at a proximal side of the shaft 30, and the hub 40 may be provided with a fluid inlet 50 that is connected to the flow channel of the fluid supplied to the interior of the balloon 20.

The shaft 30 and the hub 40 may be joined by, for example, adhesive bonding or welding. Of these, the shaft 30 and the hub 40 may be joined by adhesive bonding. The adhesive bonding of the shaft 30 and hub 40 can increase the bonding strength of the shaft 30 and hub 40 to increase durability of the balloon catheter 10 when the materials forming the shaft 30 and hub 40 are different, for example, in a case where the shaft 30 is made of material having high flexibility and the hub 40 is made of material having high stiffness.

Although not shown in the figures, one or more embodiments of the present invention are also applicable to a so-called over-the-wire type balloon catheter that has a guidewire lumen extending from the distal end to the proximal end of the shaft. In the case of the over-the-wire type, the inflation lumen and the guidewire lumen may extend to a hub positioned at the proximal side, and the proximal openings of each lumen may be provided in the hub having a bifurcated structure.

In the case of the rapid-exchange type catheter, the outer wall of the distal shaft 31 and/or the proximal shaft 32 may be coated as appropriate, or both the distal shaft 31 and the proximal shaft 32 may be coated. In the case of the over-the-wire type catheter, the outer wall of the outer shaft may be coated as appropriate.

The coating can be a hydrophilic or hydrophobic coating, depending on the purpose, and can be applied by dipping the shaft 30 into a hydrophilic or hydrophobic coating agent, applying a hydrophilic or hydrophobic coating agent to the outer wall of the shaft 30, or coating the outer wall of the shaft 30 with a hydrophilic or hydrophobic coating agent. The coating agent may contain medical agents and additives.

Hydrophilic coating agents include hydrophilic polymers such as polyvinyl alcohol, polyethylene glycol, polyacrylamide, polyvinyl pyrrolidone, methyl vinyl ether maleic anhydride copolymer, and hydrophilic coating agents made of any combination thereof.

Hydrophobic coating agents include polytetrafluoroethylene (PTFE), ethylene-propylene fluoride (FEP), perfluoroalkoxy alkane (PFA), silicone oil, hydrophobic urethane resin, carbon coat, diamond coat, diamond-like carbon (DLC) coating, ceramic coating, and substances with low surface free energy terminated with an alkyl group or a perfluoroalkyl group.

The present application claims priority based on Japanese Patent Application No. 2022-184271 filed on Nov. 17, 2022. All the contents described in Japanese Patent Application No. 2022-184271 filed on Nov. 17, 2022 are incorporated herein by reference.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

### DESCRIPTION OF REFERENCE SIGNS

10: balloon catheter
20: balloon for balloon catheter
20a: first layer
20A: first-layer-rich portion
20b: second layer
20B: second-layer-rich portion
20C: centroid of outer edge of balloon
20M: balloon film
30: shaft
31: distal shaft
32: proximal shaft
40: hub
50: fluid inlet
60: inner shaft
61: guidewire port
70: tip member
80: marker
200: parison
200a: first layer of parison
200b: second layer of parison
201: first end of parison
202: second end of parison
205: lumen of parison
250: parison mold
251: first tubular member
252: second tubular member
253: third tubular member
300: mold
300C: mold straight tubular part
300S: mold sleeve part
300T: mold tapered part
305: lumen of mold

1. A balloon for a balloon catheter having a longitudinal axis direction, a radial direction-nd a circumferential direction, comprising:
a balloon film including a first layer and a second layer composed of a material having a Shore D hardness higher than that of the first layer;
the first layer and the second layer being disposed over the entire 360° in the circumferential direction; and
the second layer being located on an outer side of the first layer in the radial direction,
wherein:
a variation rate ((|Tx−Ta|/Ta)×100) of a film thickness Tx of the balloon film at any position x in the circumferential direction with respect to an average film thickness Ta of the balloon film in a cross-section perpendicular to the longitudinal axis direction is 15% or less;
an average film thickness T1a of the first layer is 2 μm or more; and
the balloon comprises, in a cross-section perpendicular to the longitudinal axis direction, a position X1 at which a variation rate ((|T1x−T1a|/T1a)×100) of a film thickness T1x of the first layer at the position X1 in the circumferential direction with respect to the average film thickness T1a of the first layer is 20% or more.

2. The balloon for the balloon catheter according to claim 1, wherein:
in a cross-section perpendicular to the longitudinal axis direction, the balloon has a first sector, a second sector, and a third sector obtained by dividing the 360° in the circumferential direction into three sectors of 120° each; and
in each sector, the balloon comprises a position X1 at which a variation rate ((|T1x−T1a|/T1a)×100) of the film thickness T1x of the first layer at the position X1 in the circumferential direction with respect to the average film thickness T1a of the first layer is 20% or more.

3. The balloon for the balloon catheter according to claim 2, wherein, in each of the first sector, the second sector, and the third sector, the balloon comprises:
at least one first-layer-rich portion in which the film thickness T1x of the first layer is more than 50% of a film thickness Tx1 of the balloon film at the position X1 in the circumferential direction; and
at least one second-layer-rich portion in which a film thickness T2x of the second layer at a position X2 in the circumferential direction is more than 50% of a film thickness Tx2 of the balloon film at the position X2.

4. The balloon for the balloon catheter according to claim 3, wherein, in the circumferential direction of each of the sectors, the at least one first-layer-rich portion comprises a plurality of first-layer-rich portions, and the at least one second-layer-rich portion comprises a plurality of second-layer-rich portions, wherein:
a total range in which the first-layer-rich portions are arranged is 30° or more out of the 120° in the circumferential direction of each sector;
a total range in which the second-layer-rich portions are arranged is 30° or more out of the 120° in the circumferential direction of each sector; and
each of the first-layer-rich portions and each of the second-layer-rich portions are alternately arranged in the circumferential direction.

5. The balloon for the balloon catheter according to claim 1, wherein the balloon comprises:
at least one first-layer-rich portion in which the film thickness T1x of the first layer is more than 50% of a film thickness Tx1 of the balloon film at the position X1 in the circumferential direction; and
at least one second-layer-rich portion in which a film thickness T2x of the second layer at a position X2 in the circumferential direction is more than 50% of a film thickness Tx2 of the balloon film at the position X2, wherein:
in the circumferential direction, the at least one first-layer-rich portion comprises a plurality of first-layer-rich portions, and the at least one second-layer-rich portion comprises a plurality of second-layer-rich portions;
a range in which each of the first-layer-rich portions is arranged and a range in which each of the second-layer-rich portions is arranged are 15° or more, respectively, out of 360° in the circumferential direction of the balloon film; and
each of the first-layer-rich portions and each of the second-layer-rich portions are alternately arranged in the circumferential direction.

6. A balloon catheter comprising the balloon according to claim 1.
